(19) United States
(12) Patent Application Publication
(10) Pub. No.: US 2026/0137932 A1
(43) Pub. Date: May 21, 2026

(54) MICROCURRENT DEVICES

(71) Applicant: Birch Strategic Capital, LLC, Laguna Beach, CA (US)

(72) Inventor: Toni Battaglia, North Palm Beach, FL (US)

(21) Appl. No.: 18/953,257

(22) Filed: Nov. 20, 2024

(51) Int. Cl. A61N 1/32

(52) U.S. Cl. CPC A61N 1/328

(57) ABSTRACT

The present disclosure provides a dermal stimulation device and methods of use thereof. The dermal stimulation device comprises a removable head piece and an elongate base member. The elongate base member comprises a proximal portion comprising one or more conducting surfaces, a circumferential rim configured for engaging with the removable head piece, and an actuatable element configured to actuate one or more electrical components housed within the elongate handle. The removable head piece comprises a plurality of light emitting elements and a distal flange configured for engaging with the circumferential rim of the elongate base member, wherein engaging elongate base member with the removable head piece causes the elongate base member to be in electrical communication with the removable head piece.

### TECHNICAL FIELD

The present invention provides microcurrent devices and methods for stimulating the skin.

### BACKGROUND

Skin care tools provide many benefits for overall skin health, skin aesthetic appearance, and overall wellness. Many at-home skin care tools are available to consumers for enhancing their skin health and overall wellness as well as augmenting to an overall self-care practice or routine. Skin care tools can include facial rollers, at-home light therapy applicators, mechanical wave generating devices for applying mechanical vibrations to the skin, microcurrent devices for applying microcurrents to the skin, and the like.

Facial tools for administering light therapy can also offer overall wellness and skin health benefits. Red light therapy may work in skin health to: stimulate collagen production, which gives skin its structure, strength and elasticity; increase fibroblast production, which makes collagen. Collagen is a component of connective tissue that builds skin; increase blood circulation to the tissue; reduce inflammation in cells, among other things. It is currently being used clinically to improve wound healing, reduce stretch marks, reduce wrinkles, fine lines and age spots, improve facial texture, improve skin conditions like psoriasis, rosacea, and eczema, improve scar, improve sun-damaged skin, improve acne, and the like.

Microcurrent facials are cosmetic treatments using a device to deliver small currents of electricity to the skin. The use of microcurrent stimulation is believed to offer many benefits including promoting wound healing, reducing inflammation, boosting circulation, and improving muscle function.

While having multiple tools available provide useful options for skin care wellness, there is a need for multifunctional tools that can offer time-saving benefits, greater user convenience, and multiple therapeutic benefits.

### SUMMARY

In meeting the long-felt needs described above, the present disclosure provides a multifunctional dermal stimulation device for applying a light therapy to the skin and also for applying a microcurrent to the skin. In certain aspects, the dermal stimulation device as contemplated herein can include a removable head piece and an elongate base member. The elongate base member can include a proximal portion having one or more conducting surfaces; a circumferential rim configured for engaging with the removable head piece; and an actuatable element configured to actuate one or more electrical components housed within the elongate handle. The removable head piece can include a plurality of light emitting elements. The removable head can include a distal flange configured for engaging with the circumferential rim of the elongate base member. In certain aspects, engaging the elongate base member with the removable head piece causes the elongate base member to be in electrical communication with the removable head piece.

In some embodiments, the one or more conducting surfaces are configured for emitting a microcurrent. The microcurrent can include a current in the range of from about 0.5 μA to about 1 mA.

In some embodiments, the plurality of lights is housed beneath a translucent surface of the removable head piece, on an outer surface of the removable head piece, and/or a combination thereof. The plurality of light emitting elements can include an amount in the range of from about 2 to about 50 light emitting elements. In some embodiments, the light emitting elements can be positioned so that the light emitting elements are equally spaced and centered along the central axis of the roller head. In some embodiments, the light emitting elements can include light having an emission wavelength in the range of from about 620 nm to about 750 nm, preferably in the range of from about 620 nm to about 670 nm. In some embodiments, the light emitting elements can be configured for delivering an intensity dose in the range of from about 0.1 J/cm² to about 500 J/cm². In some embodiments, the light emitting elements can deliver a pulsed light. In some embodiments, the light emitting elements can deliver a continuous light.

The elongate base can include one or more electrical contacts and the removable head piece comprise one or more electrical contacts, such that when one or more of the electrical contacts of the elongate base engages with one or more of the electrical contacts of the removable head, an electrical current is conducted.

In some embodiments, the actuatable element can be configured to reversibly actuate one or more of the light emitting elements. In some embodiments, the actuatable element can be configured to reversibly actuate a microcurrent applied by the one or more conducting surfaces. In some embodiments, the actuatable element can include one or more visual indications corresponding to one or more actuation modes including, for example, an off mode, a continuous microcurrent mode, one or more pulsed microcurrent modes, a continuous light mode, and/or one or more pulsed light modes.

In some embodiments, the one or more conducting surfaces can be configured for emitting a microcurrent synchronously.

In certain aspects, the present disclosure provides methods for stimulating the skin of a subject. The methods can include the steps of: contacting a portion of the skin of the subject with the dermal stimulation device; and actuating the dermal stimulation device to emit a light signal, a microcurrent signal, and/or a combination thereof for a duration of time.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various aspects discussed in the present document. In the drawings:

FIG. 1A depicts an exemplary dermal stimulation device as contemplate by the present disclosure.

FIG. 1B depicts an expanded view of an exemplary dermal stimulation device as contemplate by the present disclosure with the removable head piece separate from the elongate base member.

FIG. 2 depicts a side view of an exemplary dermal stimulation device as contemplated by the present disclosure where the head piece is engaged with the elongate base member.

FIG. 3 depicts another side view of an exemplary dermal stimulation device as contemplated by the present disclosure where the head piece is engaged with the elongate base member.

FIGS. 4 and 5 depict a front and back side view of an exemplary dermal stimulation device as contemplated by the present disclosure with the head piece engaged with the elongate base member.

FIG. 6 depicts a top view of the dermal stimulation device with the head piece engaged with the elongate base member.

FIG. 7 depicts a bottom view of the dermal stimulation device.

FIG. 8 depicts a perspective view of the elongate base member of the dermal stimulation device.

FIG. 9 depicts a side view of the elongate base member of the dermal stimulation device.

FIG. 10 depicts a side view of the elongate base member of the dermal stimulation device.

FIGS. 11 and 12 depict a front and back side view of the elongate base member of an exemplary dermal stimulation device as contemplated by the present disclosure.

FIG. 13 depicts a top view of the elongate base member of the dermal stimulation device.

FIG. 14 depicts a bottom view of the elongate base member of the dermal stimulation device.

FIG. 15 depicts a perspective view of the removable head piece of an exemplary dermal stimulation device.

FIG. 16 depicts a bottom view of the removable head piece of an exemplary dermal stimulation device.

FIG. 17 depicts an exemplary method as contemplated by the present disclosure.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

As used herein, the term “microcurrent” refers to electrotherapy applied as a series of electrical currents of less than about 1 mA.

### DETAILED DESCRIPTION

### Dermal Stimulation Device

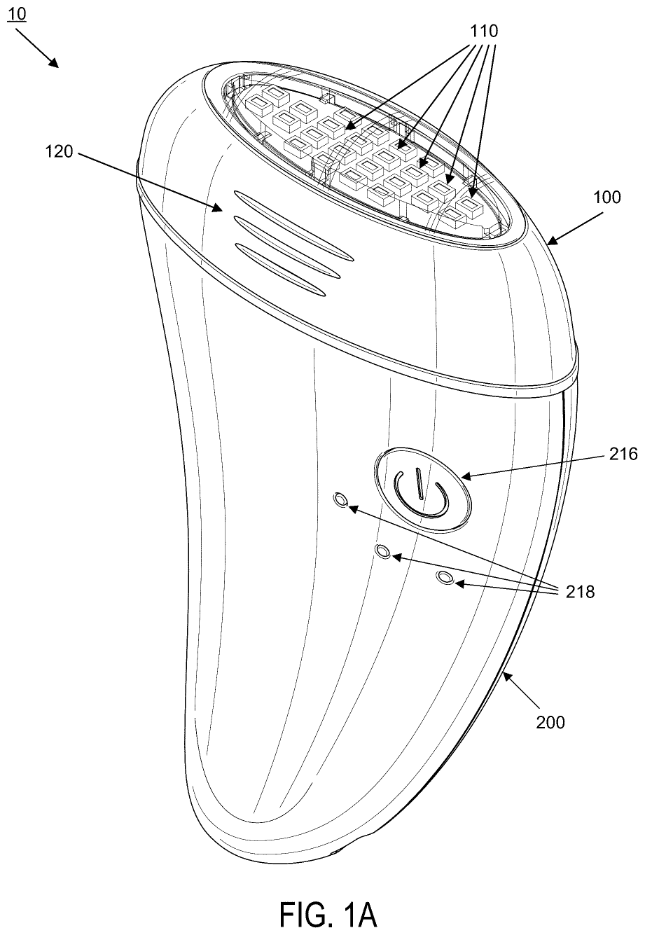

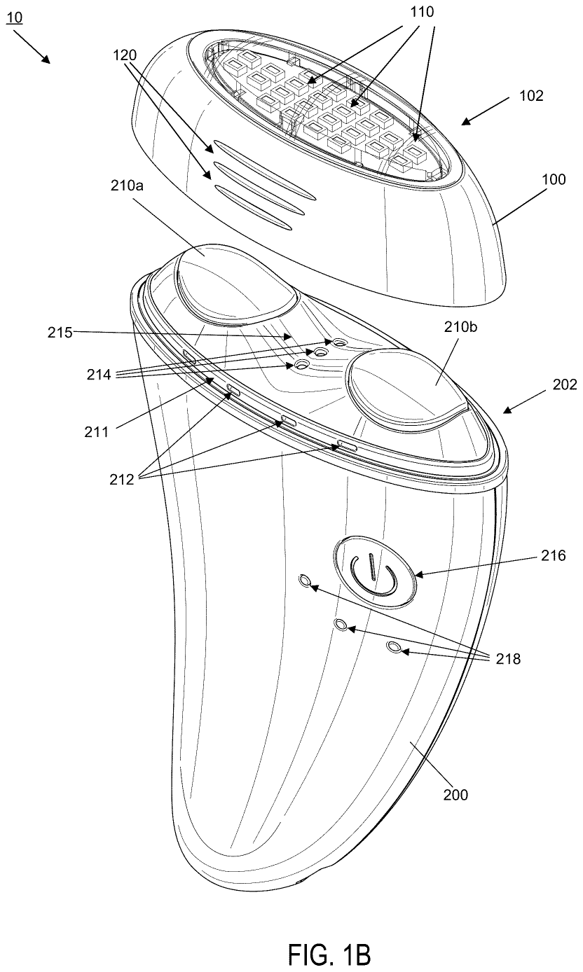

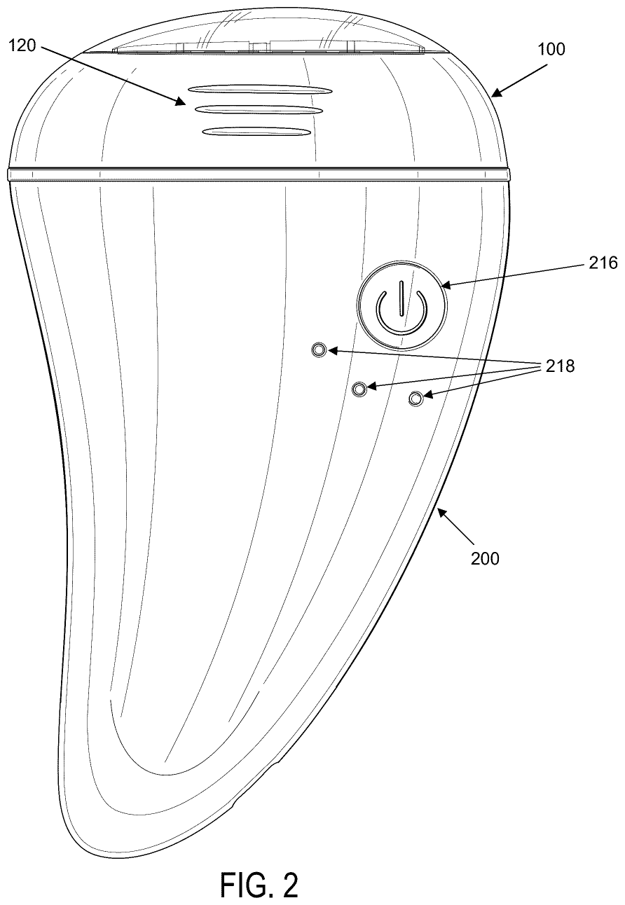

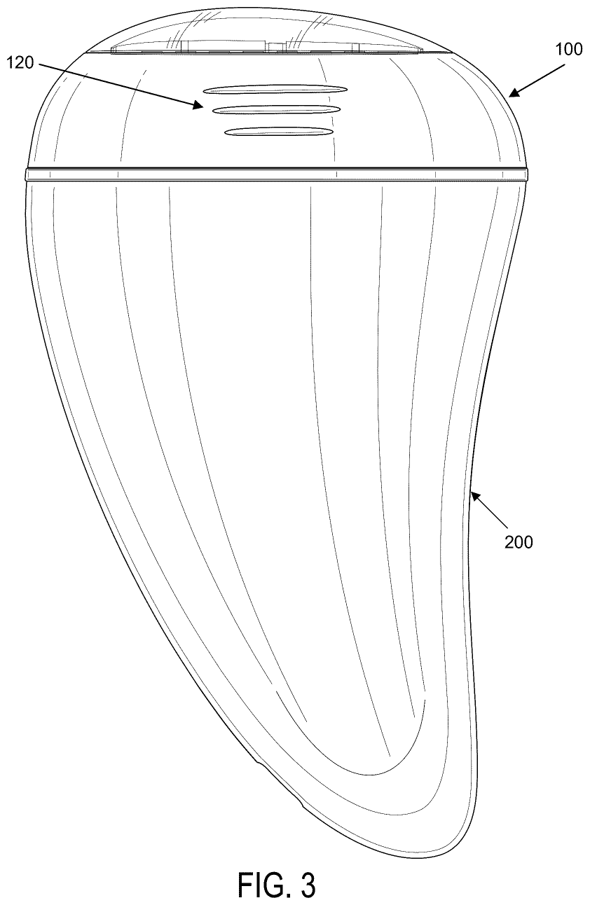

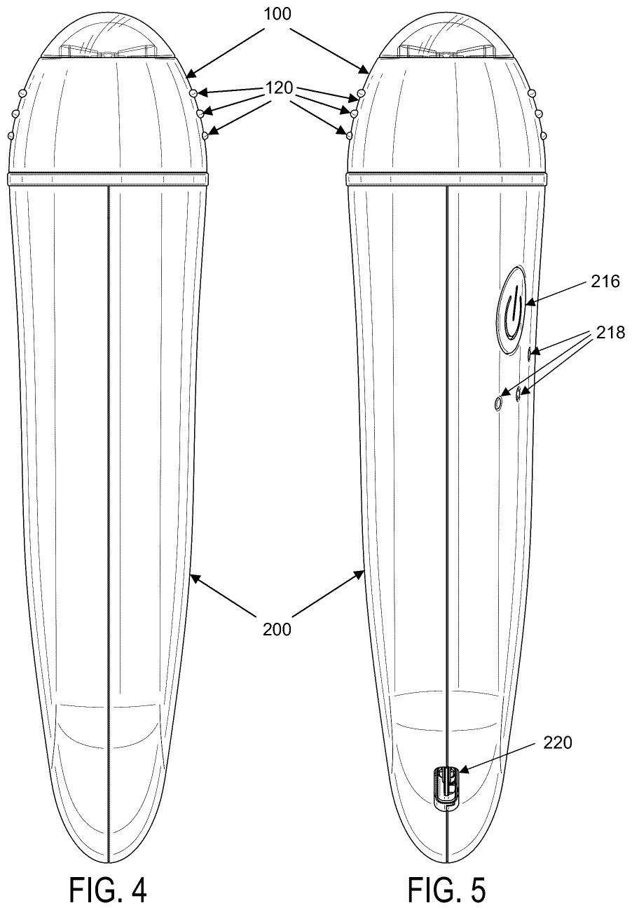

The present disclosure provides dermal stimulation devices and methods for stimulating the skin in a subject in need thereof. The skin can include any suitable surface of the body including for example, the face, neck, chest, hands, arms, legs, feet, torso, abdomen, back, and the like. The dermal stimulation device can include a hand-held device. Referring now to FIGS. 1A and 1B, the dermal stimulation device 10 can include a removable head piece 100 and an elongate base member 200. The elongate base member 200 can include a proximal portion 202 having one or more conducting surfaces 210a/210b. The one or more conducting surfaces 210a/210b can be constructed of any suitable conducting material. The one or more conducting surfaces 210a/210b can be configured for emitting a microcurrent. The microcurrent can include a current in the range of from about 0.5 μA to about 1 mA. For example, the microcurrent can include a current in the range of from about 0.5 μA to about 1 μA, from about 1 μA to about 5 μA, from about 5 μA to about 10 μA, from about 10 μA to about 50 μA, from about 50 μA to about 100 μA, from about 100 μA to about 500 μA, from about 500 μA to about 1 mA, including any and all increments therebetween. The microcurrent can be emitted in one or more stimulation sequence modes. The one or more stimulation sequence modes can include, for example, a continuous stimulation sequence, an interval pulsed stimulation sequence, or one or more combinations thereof. The one or more conducting surfaces can be configured for emitting a microcurrent synchronously.

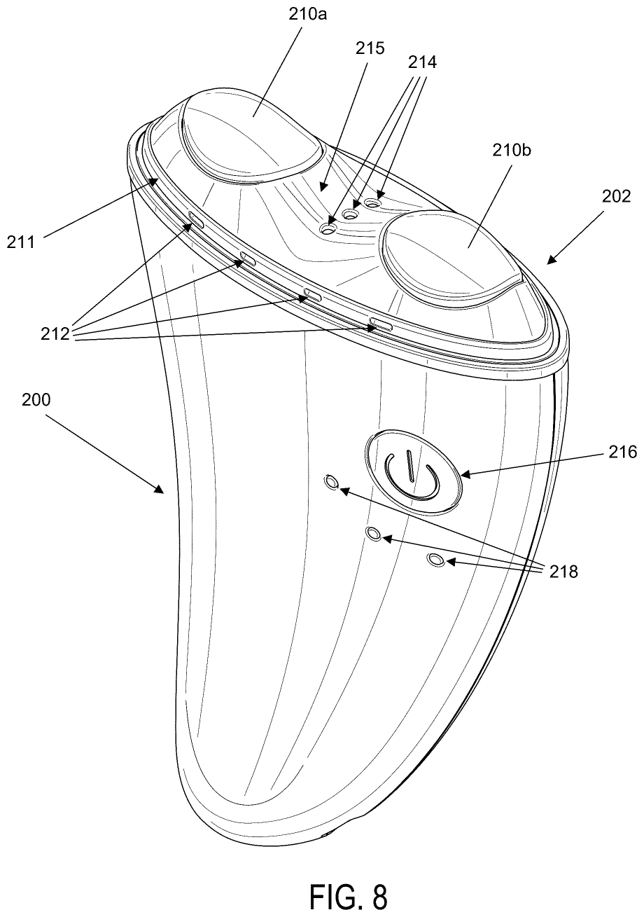

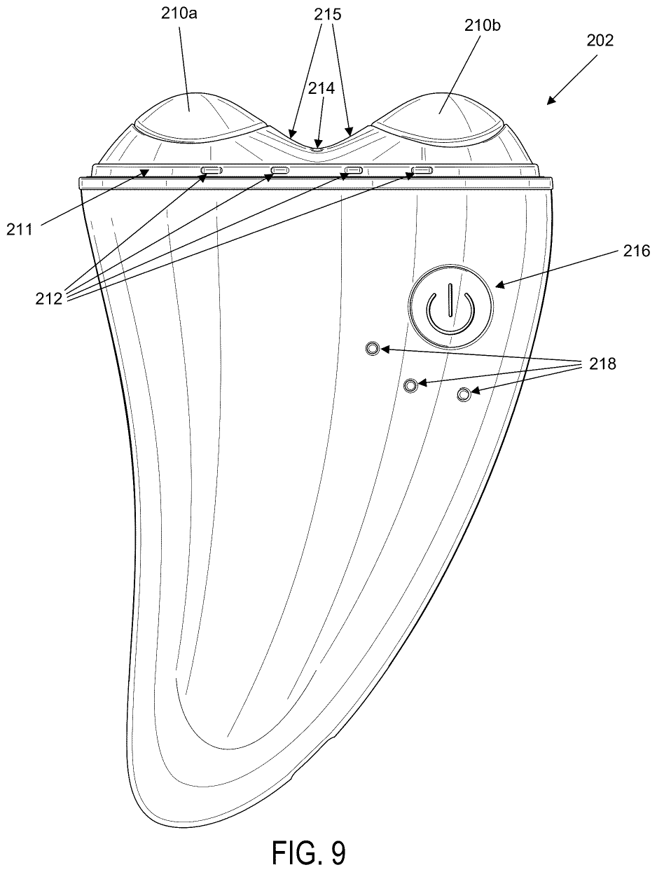

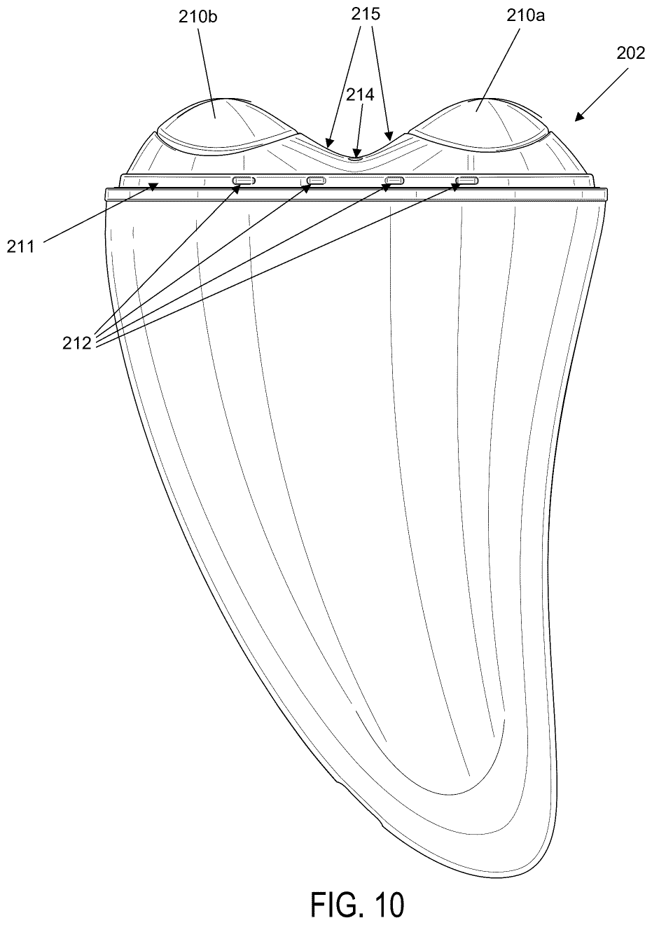

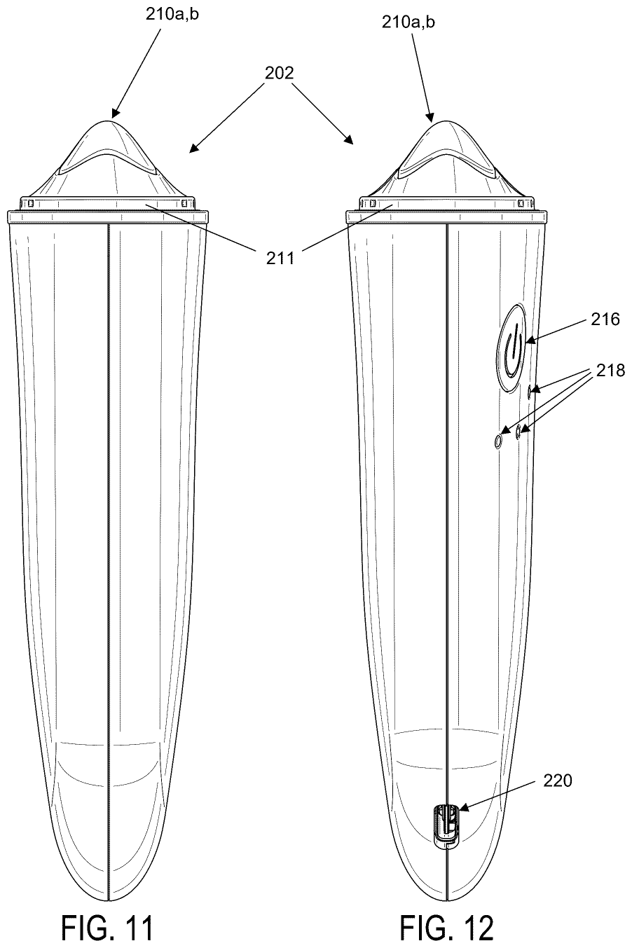

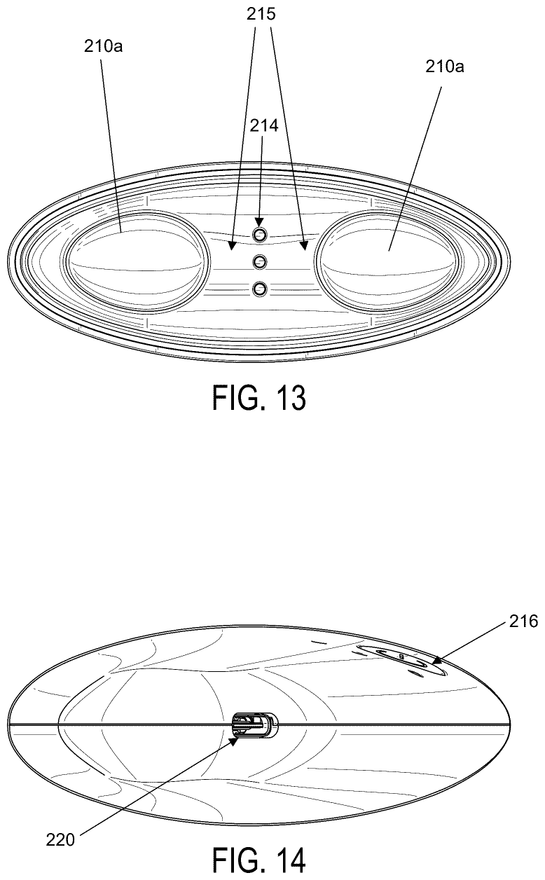

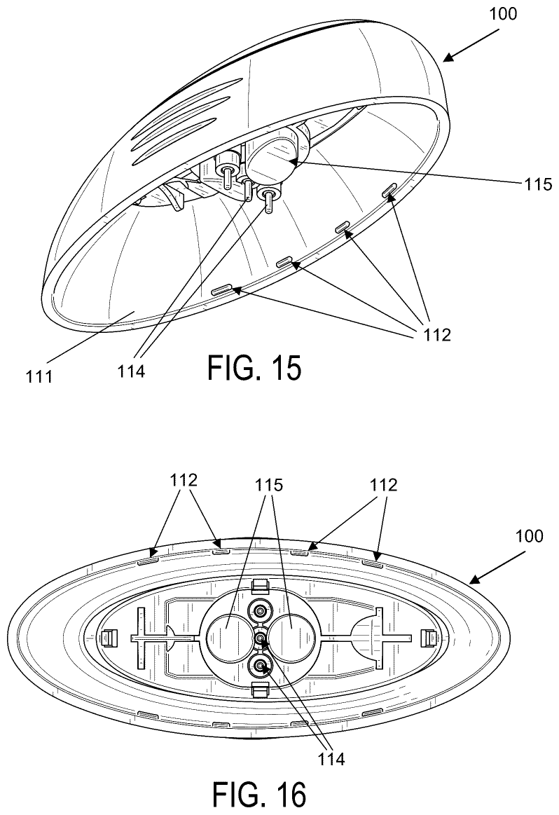

The elongate base member 200 can include a circumferential rim 211 having one or more indentations 212 positioned on the outer surface of the circumferential rim 211. The one or more indentations 212 can be configured for engaging with one or more protrusions 112 (shown in FIG. 15) on an inner surface 111 of the removable head piece 100. Alternatively, in some embodiments, the circumferential rim 211 has one or more protrusions that engage with one or more indentations on the inner surface 111 of the removable headpiece 100. The elongate base member 200 can include one or more base member magnetic elements 215 (shown in FIGS. 1B, 8, 9, 10, 13) under the surface of the proximal end. The one or more base member magnetic elements 215 can engage with one or more head piece magnetic elements 115 (shown in FIGS. 15 and 16) for physically securing removable head piece 100 to elongate base member 200.

The elongate base member 200 can include one or more electrical contacts 214 and the removable head piece can include one or more electrical pins 114, (shown in FIGS. 15 and 16), such that when one or more of the electrical contacts 214 of the elongate base member 200 engages with one or more of the electrical pins 114 of the removable head, an electrical current is conducted. That is, engaging the elongate base member 200 with the removable head piece 100 causes the elongate base member 200 to be in electrical communication with the removable head piece 100.

The elongate base member 200 can include a body constructed of one or more nonconductive materials. For example, the body of elongate base member 200 can be constructed of one or more sterilizable polymeric materials including, for example, a polypropylene, a polycarbonate, a polymethyl methacrylate, a polyvinyl chloride, a polystyrene, a polyethylene terephthalate glycol, an acrylonitrile butadiene styrene, or one or more combinations thereof.

The elongate base member 200 can include an actuatable element 216. The actuatable element 216 can be configured to actuate one or more electrical components housed within the elongate handle. The actuatable element 216 can be configured to reversibly actuate one or more of the light emitting elements. In some embodiments, the actuatable element 216 can be configured to reversibly actuate a microcurrent applied by the one or more conducting surfaces. The actuatable element 216 can include one or more visual indicators 218 (e.g., lights, light emitting diodes) corresponding to one or more actuation modes including for example, an off mode, a continuous microcurrent mode, one or more pulsed microcurrent modes, a continuous light mode, one or more pulsed light modes, or one or more combinations thereof.

The elongate base member 200 can house one or more electronic elements including, for example, one or more controllers including microcontrollers, batteries, power supplies, wired receivers, wireless receivers, circuits, and the like. The one or more electronic elements may be powered by an internal power source, an external power source, or a combination of both. The external power source can engage with the one or more electronic elements via a power port 220. The power port 220 can include, for example a USB-A port, a USB-B port, a USB-C port, an AC power supply pin receiver port, or other suitable power port.

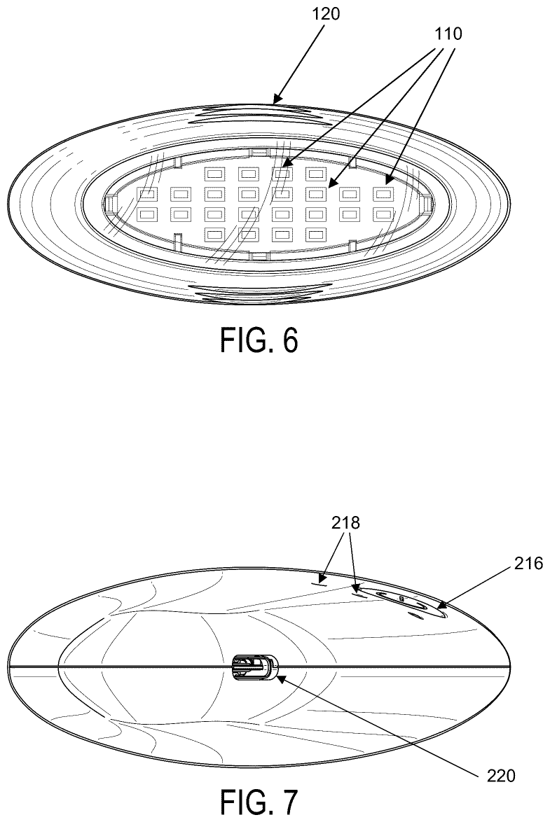

As shown in FIGS. 1A, 1B and 6, the removable head piece 100 can include a plurality of light emitting elements 110. The plurality of light emitting elements 110 can be housed, for example, beneath a translucent surface of the removable head piece, on an outer surface of the removable head piece, and/or a combination thereof. The distal inner rim of removable head piece 100 can include one or more ridges (shown in FIG. 15) for engaging with the one or more protrusions on the proximal outer rim of elongate base member 200. The removable head piece 100 can also include one or more ridges 120 for gripping the head piece when placing or removing the head piece 100 from the elongate base member 200.

The proximal portion 102 of the removable head piece 100 can be constructed of one or more sterilizable polymeric materials including, for example, a polypropylene, a polycarbonate, a polymethyl methacrylate, a polyvinyl chloride, a polystyrene, a polyethylene terephthalate glycol, an acrylonitrile butadiene styrene, or one or more combinations thereof.

The plurality of light emitting elements 110, shown in FIGS. 1B and 6, can include an amount of light emitting elements in the range of from about 2 to about 50 light emitting elements. In some embodiments, the plurality of light emitting elements can include a single light element. In some embodiments, the plurality of light emitting elements includes a number of light in the range of from about 2 to about 4 light emitting elements, from about 4 to about 6 light emitting elements, from about 6 to about 8 light emitting elements, from about 8 to about 10 light emitting elements, from about 10 to about 12 light emitting elements, from about 12 to about 14 light emitting elements, from about 14 to about 16 light emitting elements, from about 16 to about 18 light emitting elements, from about 18 to about 20 light emitting elements, from about 20 to about 22 light emitting elements, from about 22 to about 24 light emitting elements, from about 24 to about 26 light emitting elements, from about 26 to about 28 light emitting elements, from about 28 to about 30 light emitting elements, from about 30 to about 32 light emitting elements, from about 32 to about 34 light emitting elements, from about 34 to about 36 light emitting elements, from about 36 to about 38 light emitting elements, from about 38 to about 40 light emitting elements, from about 40 to about 42 light emitting elements, from about 42 to about 44 light emitting elements, from about 44 to about 46 light emitting elements, from about 46 to about 48 light emitting elements, from about 48 to about 50 light emitting elements, and so on. The light emitting elements can be arranged in one or more patterns.

The light emitting elements 110 can emit light having a wavelength in the range of from about 600 nm to about 800 nm. In some embodiments, the light emitting elements 110 can emit light having a wavelength in the range of from about 620 nm to about 750 nm. In some embodiments, the light emitting elements 110 can emit light having a wavelength in the range of from about 630 nm to about 700 nm. In some embodiments, the light emitting elements 110 comprise light having an emission wavelength preferably in the range of from about 620 nm to about 670 nm.

The light emitting elements 110 can include any suitable light emitting element as understood in the art including, for example, one or more light emitting diodes (LEDS).

The light emitting elements 110 can be positioned so that the light emitting elements 110 are equally spaced and centered along a central axis of the removable head piece. The light emitting elements can include light having an emission wavelength in the range of from about 620 nm to about 750 nm, preferably in the range of from about 620 nm to about 670 nm.

In some embodiments, the light emitting elements 110 are configured for delivering an intensity dose in the range of from about 0.1 J/cm² to about 500 J/cm². For example, the intensity dose can include up to about 0.1 J/cm², from about 0.1 J/cm² to about 1 J/cm², from about 1 J/cm² to about 10 J/cm², from about 10 J/cm² to about 50 J/cm², from about 50 J/cm² to about 100 J/cm², from about 100 J/cm² to about 200 J/cm², from about 200 J/cm² to about 300 J/cm², from about 300 J/cm² to about 400 J/cm², from about 400 J/cm² to about 500 J/cm², including any and all increments therebetween

In some embodiments, the light emitting elements 110 are configured for delivering an irradiance in the range of from about 1 mW/cm² to about 500 mW/cm². For example, the light emitting elements 110 can deliver an irradiance of from about 1 mW/cm² to about 10 mW/cm², from about 10 mW/cm² to about 50 mW/cm², from about 50 mW/cm² to about 100 mW/cm², from about 100 mW/cm² to about 150 mW/cm², from about 150 mW/cm² to about 200 mW/cm², from about 200 mW/cm² to about 250 mW/cm², from about 250 mW/cm² to about 300 mW/cm², from about 300 mW/cm² to about 350 mW/cm², from about 350 mW/cm² to about 400 mW/cm², from about 400 mW/cm² to about 450 mW/cm², from about 450 mW/cm² to about 500 mW/cm² including any and all increments therebetween. In preferred embodiments, the light emitting elements 110 are configured for delivering an intensity dose in the range of from about 50 mW/cm² to about 70 mW/cm².

In some embodiments, the light emitting elements 110 deliver a pulsed light. The pulsed light can be pulsed according to one or more patterns comprising one or more combinations of pulse durations and pulse frequencies. In some embodiments, the light emitting elements 110 can deliver a continuous light. The actuation element 216 can allow for changing between pulsed light, continuous light, and/or other states.

### Methods

In certain aspects, the present disclosure provides methods 1700 for stimulating the skin of a subject.

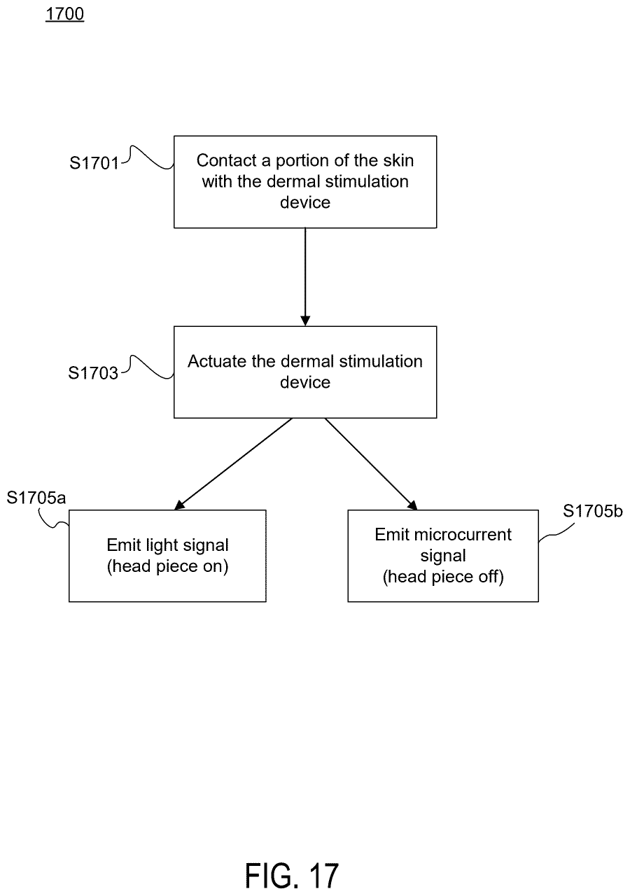

Referring now to FIG. 17, embodiments of method 1700 include step S1701 which includes contacting a portion of the skin of the subject with the dermal stimulation device as described herein. The portion of skin can include any suitable portion of skin including for example, the face, neck, chest, hands, arms, legs, feet, torso, abdomen, back, and the like.

Embodiments of methods 1700 can include step S1703, which includes actuating the dermal stimulation device to emit a light signal, a microcurrent signal, or a combination thereof for a duration of time. The dermal stimulation device 10 can be actuated by depressing the actuatable element 216. In some embodiments, depressing the actuatable element 216 one time actuates a first low setting. The first low setting can be indicated by one or more of the visual indications 218 being illuminated. In some embodiments, depressing the actuatable element 216 two times in rapid succession can actuate a second intermediate setting. In some embodiments, the second intermediate setting can be indicated by two of visual indications 218 being illuminated. In some embodiments, depressing the actuatable element 216 three times in rapid succession can actuate a third high setting. In some embodiments, the third high setting can be indicated by three of visual indications 218 being illuminated. The duration of time can include a duration of including for example up to about 30 seconds; a duration of from about 30 seconds to about 1 minute, a duration of from about 1 minute to about 2 minutes, a duration of from about 2 minutes to about 4 minutes, a duration of from about 4 minutes to about 6 minutes, a duration of from about 6 minutes to about 8 minutes, a duration of from about 8 minutes to about 10 minutes, and so on, including any and all increments therebetween.

Embodiments of step S1703 include actuating the dermal stimulation device with the removable head piece engaged with the elongate base member (i.e., the head piece on the base, shown in FIG. 1a). In such embodiments, the methods include step S1705a, such that when the actuatable element 216 is actuated, the light element(s) 110 are actuated to emit light. That is, when one or more pins 114 of removeable head piece engage with one or more electrical contacts 214 of elongate base member 200, one or more microcontrollers or switches housed within the body of elongate base member 200 cause power to be directed to the light emitting elements 110 of removable headpiece 100. In some embodiments, when light emitting elements 110 are receiving power, conducting surfaces 210a/210b are not receiving power. That is, power to conducting surfaces 210a/210b is interrupted or diverted. That is, when the head piece 100 is engaged with elongate base member 200, light emitting elements 110 are actuated to emit light and conducting surfaces 210a/210b are not actuated. The light can include light having an emission wavelength in the range of from about 620 nm to about 750 nm, preferably in the range of from about 620 nm to about 670 nm. The light emitting elements 110 can be actuated to deliver an irradiance in the range of from about 1 mW/cm² to about 500 mW/cm². For example, the light emitting elements 110 can be actuated to deliver an irradiance of from about 1 mW/cm² to about 10 mW/cm², from about 10 mW/cm² to about 50 mW/cm², from about 50 mW/cm² to about 100 mW/cm², from about 100 mW/cm² to about 150 mW/cm², from about 150 mW/cm² to about 200 mW/cm², from about 200 mW/cm² to about 250 mW/cm², from about 250 mW/cm² to about 300 mW/cm², from about 300 mW/cm² to about 350 mW/cm², from about 350 mW/cm² to about 400 mW/cm², from about 400 mW/cm² to about 450 mW/cm², from about 450 mW/cm² to about 500 mW/cm² including any and all increments therebetween. In preferred embodiments, the light emitting elements are actuated to deliver an irradiance in the range of from about 50 mW/cm² to about 70 mW/cm².

Embodiments of step S1703 include actuating the dermal stimulation device with the removable head piece disengaged from the elongate base member (i.e., the head piece off the base, shown in FIG. 1b). In such embodiments, the methods include step S1705b, such that when the actuatable element 216 is actuated, the one or more conducting surfaces 210a/210b are actuated to emit a microcurrent across a segment of the skin or underlying tissue of the subject. In some embodiments, the microcurrent travels from a first conducting surface 210a to a second conducting surface 210b. The one or more conducting surfaces 210a/210b can be actuated to emit a microcurrent including a current in the range of from about 0.5 μA to about 1 mA. For example, the microcurrent can include a current in the range of from about 0.5 μA to about 1 μA, from about 1 μA to about 5 μA, from about 5 μA to about 10 μA, from about 10 μA to about 50 μA, from about 50 μA to about 100 μA, from about 100 μA to about 500 μA, from about 500 μA to about 1 mA, including any and all increments therebetween. The one or more conducting surfaces 210a/210b can be actuated to emit a microcurrent in one or more stimulation sequence modes. The one or more stimulation sequence modes can include, for example, a continuous stimulation sequence, an interval pulsed stimulation sequence, or one or more combinations thereof. The one or more conducting surfaces 210a/210b can be actuated to emit a microcurrent synchronously.

### Aspects

The following Aspects are illustrative only and do not limit the scope of the present disclosure or the appended claims. Any part or parts of any one or more Aspects can be combined with any part or parts of any one or more other Aspects.
Aspect 1. A dermal stimulation device comprising a removable head piece and an elongate base member wherein: the elongate base member comprises: a proximal portion comprising one or more conducting surfaces; a circumferential rim configured for engaging with the removable head piece; and an actuatable element configured to actuate one or more electrical components housed within the elongate handle; and, the removable head piece comprises: a plurality of light emitting elements; and, a distal flange configured for engaging with the circumferential rim of the elongate base member; wherein engaging elongate base member with the removable head piece causes the elongate base member to be in electrical communication with the removable head piece.
Aspect 2. The dermal stimulation device of Aspect 1, wherein the one or more conducting surfaces are configured for emitting a microcurrent.
Aspect 3. The dermal stimulation device of Aspect 2, wherein the microcurrent comprises a current in the range of from about 0.5 μA to about 1 mA.
Aspect 4. The dermal stimulation device of Aspect 2, wherein the microcurrent is emitted in one or more stimulation sequence modes.
Aspect 5. The dermal simulation device of Aspect 4, wherein the one or more stimulation sequence modes comprise a continuous stimulation sequence, an interval pulsed stimulation sequence, or one or more combinations thereof.
Aspect 6. The dermal stimulation device of any one of Aspects 1 to 5, wherein the plurality of lights is housed beneath a translucent surface of the removable head piece, on an outer surface of the removable head piece, and/or a combination thereof.
Aspect 7. The dermal stimulation device of any one of Aspects 1 to 6, wherein the plurality of light emitting elements comprises an amount in the range of from about 2 to about 50 light emitting elements.
Aspect 8. The dermal stimulation device of any one of Aspects 1 to 7, wherein the light emitting elements are positioned so that the light emitting elements are equally spaced and centered along the central axis of the roller head.
Aspect 9. The dermal stimulation device of any one of Aspects 1 to 8, wherein the light emitting elements comprise light having an emission wavelength in the range of from about 620 nm to about 750 nm, preferably in the range of from about 620 nm to about 670 nm.
Aspect 10. The dermal stimulation device of any one of Aspects 1 to 9, wherein the light emitting elements are configured for delivering an intensity dose in the range of from about 0.1 J/cm² to about 500 J/cm².
Aspect 11. The dermal stimulation device of any one of Aspects 1 to 10, wherein the light emitting elements deliver a pulsed light.
Aspect 12. The dermal stimulation device of Aspect 11, wherein the pulsed light is pulsed according to one or more patterns comprising one or more combinations of pulse durations and pulse frequencies.
Aspect 13. The dermal stimulation device of any one of Aspects 1 to 10, wherein the light emitting elements deliver a continuous light.
Aspect 14. The dermal stimulation device of any one of Aspects 1 to 13, wherein the elongate base comprises one or more electrical contacts and the removable head piece comprise one or more electrical contacts, wherein when one or more of the electrical contacts of the elongate base engages with one or more of the electrical contacts of the removable head, an electrical current is conducted.
Aspect 15. The dermal stimulation device of any one of Aspects 1 to 14, wherein the actuatable element is configured to reversibly actuate one or more of the light emitting elements.
Aspect 16. The dermal stimulation device of Aspect 2, wherein the actuatable element is configured to reversibly actuate a microcurrent applied by the one or more conducting surfaces.
Aspect 17. The dermal stimulation device of any one of Aspects 1 to 16, wherein the actuatable element comprises one or more visual indications corresponding to one or more actuation modes comprising: an off mode, a continuous microcurrent mode, one or more pulsed microcurrent modes, a continuous light mode, and/or one or more pulsed light modes.
Aspect 18. The dermal stimulation device of any one of Aspects 1 to 17, wherein the proximal portion of the removable head piece is constructed of one or more sterilizable polymeric materials comprising a polypropylene, a polycarbonate, a polymethyl methacrylate, and/or a polyethylene terephthalate glycol.
Aspect 19. The dermal stimulation device of any one of Aspects 1 to 18, wherein the elongate base member comprises a body constructed of one or more nonconductive materials.
Aspect 20. The dermal stimulation device of Aspect 2, wherein the one or more conducting surfaces are configured for emitting a microcurrent synchronously.
Aspect 21. A method for stimulating the skin of a subject comprising: contacting a portion of the skin of the subject with the dermal stimulation device of Aspect 1 and actuating the dermal stimulation device to emit a light signal, a microcurrent signal, or a combination thereof for a duration of time.

What is claimed:

1. A dermal stimulation device comprising a removable head piece and an elongate base member wherein:
the elongate base member comprises:
a proximal portion comprising one or more conducting surfaces;
a circumferential rim configured for engaging with the removable head piece; and
an actuatable element configured to actuate one or more electrical components housed within the elongate handle; and,
the removable head piece comprises:
a plurality of light emitting elements; and,
a distal flange configured for engaging with the circumferential rim of the elongate base member;
wherein engaging elongate base member with the removable head piece causes the elongate base member to be in electrical communication with the removable head piece.

2. The dermal stimulation device of claim 1, wherein the one or more conducting surfaces are configured for emitting a microcurrent.

3. The dermal stimulation device of claim 2, wherein the microcurrent comprises a current in the range of from about 0.5 μA to about 1 mA.

4. The dermal stimulation device of claim 2, wherein the microcurrent is emitted in one or more stimulation sequence modes.

5. The dermal simulation device of claim 4, wherein the one or more stimulation sequence modes comprise a continuous stimulation sequence, an interval pulsed stimulation sequence, or one or more combinations thereof.

6. The dermal stimulation device of claim 1, wherein the plurality of light emitting elements is housed beneath a translucent surface of the removable head piece, on an outer surface of the removable head piece, and/or a combination thereof.

7. The dermal stimulation device of claim 1, wherein the plurality of light emitting elements comprises an amount in the range of from about 2 to about 50 light emitting elements.

8. The dermal stimulation device of claim 1, wherein the light emitting elements are positioned so that the light emitting elements are equally spaced and centered along the central axis of the roller head.

9. The dermal stimulation device of claim 1, wherein the light emitting elements comprise light having an emission wavelength in the range of from about 620 nm to about 750 nm, preferably in the range of from about 620 nm to about 670 nm.

10. The dermal stimulation device of claim 1, wherein the light emitting elements are configured for delivering an intensity dose in the range of from about 0.1 J/cm² to about 500 J/cm².

11. The dermal stimulation device of claim 1, wherein the light emitting elements deliver a pulsed light.

12. The dermal stimulation device of claim 11, wherein the pulsed light is pulsed according to one or more patterns comprising one or more combinations of pulse durations and pulse frequencies.

13. The dermal stimulation device of claim 1, wherein the light emitting elements deliver a continuous light.

14. The dermal stimulation device of claim 1, wherein the elongate base comprises one or more electrical contacts and the removable head piece comprise one or more electrical contacts, wherein when one or more of the electrical contacts of the elongate base engages with one or more of the electrical contacts of the removable head, an electrical current is conducted.

15. The dermal stimulation device of claim 1, wherein the actuatable element is configured to reversibly actuate one or more of the light emitting elements.

16. The dermal stimulation device of claim 2, wherein the actuatable element is configured to reversibly actuate a microcurrent applied by the one or more conducting surfaces.

17. The dermal stimulation device of claim 1, wherein the actuatable element comprises one or more visual indications corresponding to one or more actuation modes comprising: an off mode, a continuous microcurrent mode, one or more pulsed microcurrent modes, a continuous light mode, and/or one or more pulsed light modes.

18. The dermal stimulation device of claim 1, wherein the proximal portion of the removable head piece is constructed of one or more sterilizable polymeric materials comprising a polypropylene, a polycarbonate, a polymethyl methacrylate, and/or a polyethylene terephthalate glycol.

19. The dermal stimulation device of claim 1, wherein the elongate base member comprises a body constructed of one or more nonconductive materials.

20. The dermal stimulation device of claim 2, wherein the one or more conducting surfaces are configured for emitting a microcurrent synchronously.

21. A method for stimulating the skin of a subject comprising:
contacting a portion of the skin of the subject with the dermal stimulation device of claim 1, and
actuating the dermal stimulation device to emit a light signal, a microcurrent signal, or a combination thereof for a duration of time.
